(19) United States
(12) Patent Application Publication
(10) Pub. No.: US 2023/0263407 A1
(43) Pub. Date: Aug. 24, 2023

(54) MOBILE CARDIAC MONITORING DEVICE AND METHODS OF USING SAME

(71) Applicants: Dextronix, Portland (US); SR-Medizinelektronik, Stuttgart (DE)

(72) Inventors: Thomas Alexander Jacob, Portland, OR (US); Hartmut Richter, Stuttgart (DE); Scott Lewis Zellmer, Portland, OR (US)

(73) Assignees: SR-Medizinelektronik, Stuttgart (DE); Dextronix, Portland (US)

(21) Appl. No.: 17/747,230

(22) Filed: May 18, 2022

Related U.S. Application Data
(60) Provisional application No. 63/311,665, filed on Feb. 18, 2022.

(51) Int. Cl. A61B 5/0205; A61B 5/257; A61B 5/28; A61B 5/00; A61B 5/11

(52) U.S. Cl. CPC A61B 5/02055; A61B 5/257; A61B 5/28; A61B 5/0022; A61B 5/002; A61B 5/1112; A61B 5/6833; A61B 2562/0219; A61B 2560/0209; A61B 2562/0204; A61B 2562/166; A61B 2503/40

(57) ABSTRACT

A system includes a monitoring device including a housing having a top surface and a bottom surface, the monitoring device having a processor and a memory and being configured and arranged to measure cardiac data, the monitoring device having a plurality of receivers configured to accept at least one of a plurality of single-use skin electrode patches or an adapter having integrated contact electrodes.

### CROSS-REFERENCE TO RELATED APPLICATIONS

This application claims priority to U.S. Provisional Application Ser. No. 63/311,665, entitled “MOBILE CARDIAC MONITORING DEVICE AND METHODS OF USING SAME,” filed on Feb. 18, 2022, the disclosure of which is hereby incorporated in its entirety as if fully set forth herein.

### FIELD OF THE DISCLOSURE

The present disclosure relates to collecting and transmitting cardiac data for use in medical settings. More particularly the present disclosure relates to wireless heart monitors and related transmitters and accessories.

### BACKGROUND OF THE DISCLOSURE

Medical devices such as medical sensors are used both in medical institutions and at-home to monitor one or more vital signs or to observe and record patient medical data. The medical data is transported to medical personnel who are often specialists and have the expertise to analyze the medical data and to prepare a medical report associated with the medical data. In some instances, the medical personnel use specialized systems, methods, test systems, expert workstations and/or software to aid in the analysis of the patient medical data and in the preparation of a medical report which analyzes the patient medical data.

To accomplish this, multiple devices must be used and the medical data must be readily available to the medical specialists. However, often the patient and medical data are located in remote locations away from the location of an available medical specialist. One solution is for the medical specialist to travel to the remote location such as a remote medical facility, hospital, or doctor's office to gain access to the patient medical data and to analyze the report. In other cases, the medical data is obtained at a remote facility and shipped via mail or courier service to medical facility or medical specialist located at a remote location. However, these processes are both costly and time consuming and are not desirable when the timely analysis of the patient medical data is critical to providing timely medical care to patients who may have a severe or life-threatening condition.

Therefore, it is desirable to have devices, systems and methods for acquiring patient medical data at a first location for analysis by a medical specialist who is located at a remote distance.

### SUMMARY OF THE DISCLOSURE

In some examples, a system includes a monitoring device including a housing having a top surface and a bottom surface, the monitoring device having a processor and a memory and being configured and arranged to measure cardiac data, the monitoring device having a plurality of receivers configured to accept at least one of a plurality of single-use skin electrode patches or a electromechanical adapter having integrated contact electrodes that replaces the need for “traditionally used” electrode lead cables to capture such cardiac and vital sign data from a patient's body.

### BRIEF DESCRIPTION OF THE DISCLOSURE

Various embodiments of the presently disclosed devices are shown herein with reference to the drawings, wherein:

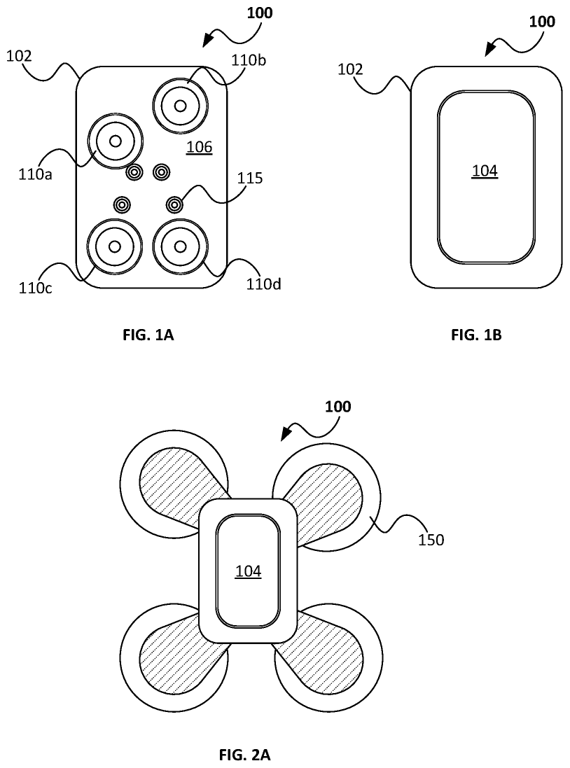

FIGS. 1A-B are schematic bottom and top views of a diagnostic monitor according to one embodiment of the present disclosure;

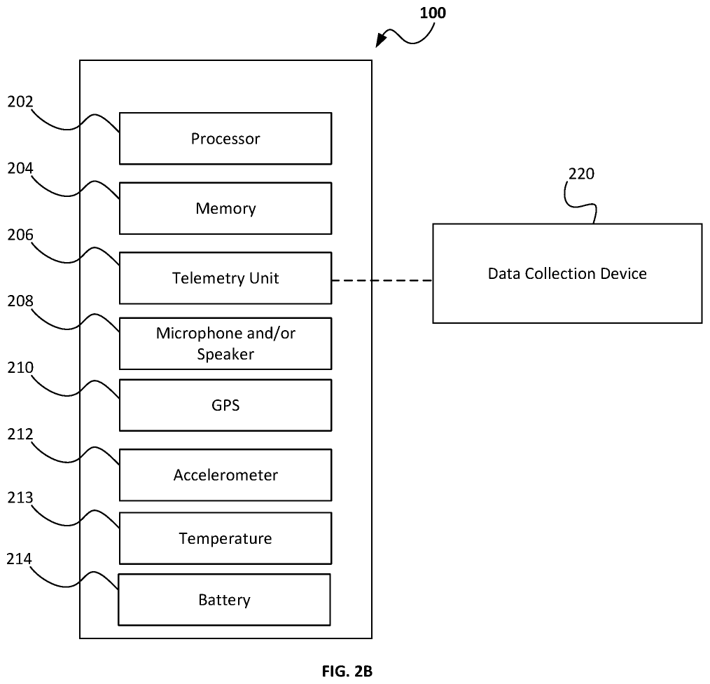

FIG. 2A is a schematic top view of the diagnostic monitoring device of FIG. 1 being coupled to disposable skin adhesive electrodes;

FIG. 2B is a schematic block diagram showing various components of a diagnostic monitoring device;

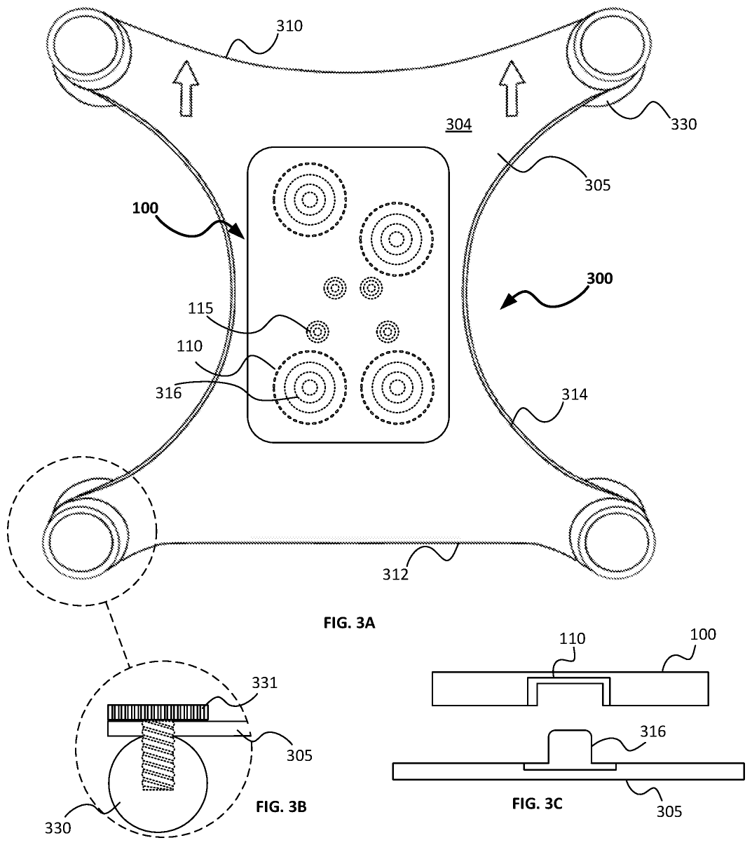

FIGS. 3A-C are a schematic top view of the diagnostic monitoring device of FIG. 1 being coupled to an adapter, and detailed views of certain portions of the adapter;

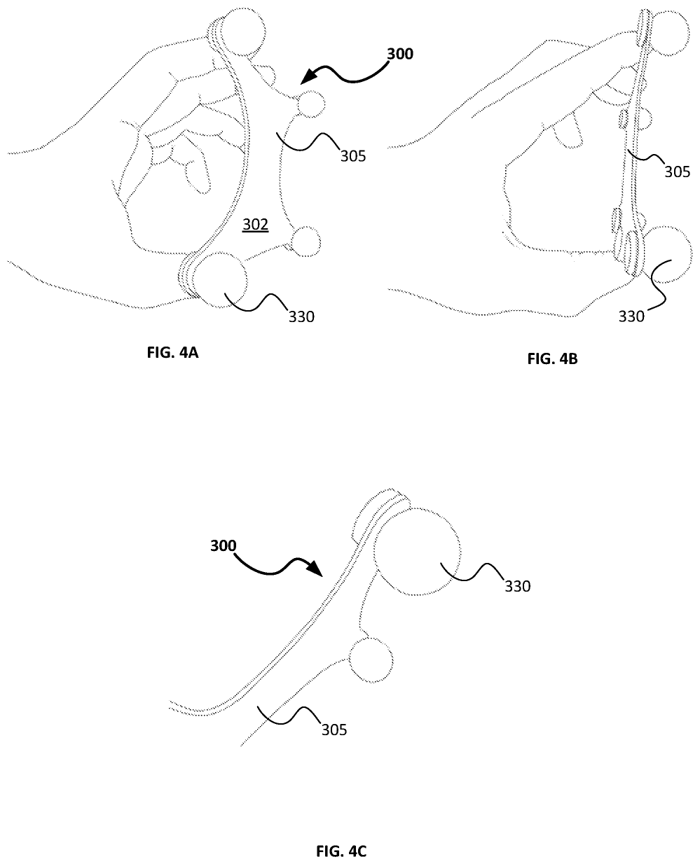

FIGS. 4A-C are schematic perspective views of one embodiment of an adapter; and

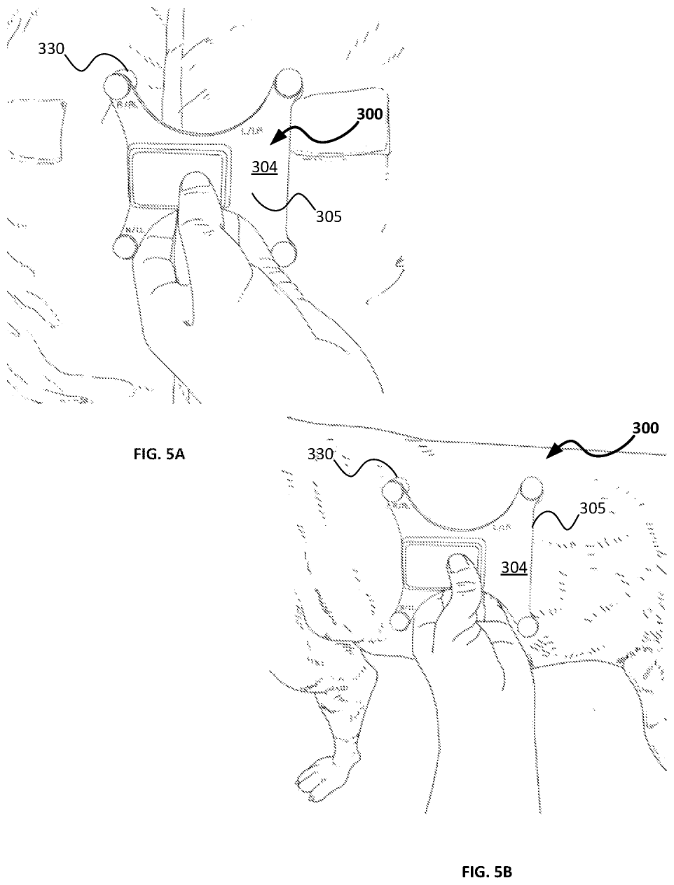

FIGS. 5A-B are schematic perspective views of the use of the diagnostic monitoring device when coupled to the adapter.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to cardiac sensors and their methods of use, conventional devices suffer from some shortcomings as described above. There therefore is a need for further improvements to the devices, systems, and methods of making and using cardiac sensors. Among other advantages, the present disclosure may address one or more of these needs.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the patient during use. The proximal end refers to the opposite end, or the end nearest the patient during use. Likewise, “tissue” or “patient” are used in the broadest sense, to refer to any tissue or substance of or within a human or animal body, and the procedures and techniques described herein may performed in vivo or in vitro.

In some examples, the term “Mobile Cardiac Telemetry” or MCT will be in connection with any sensing device capable of wireless transmission of medical heart and related data to any webserver, mobile, and stationary computer devices for diagnostic, monitoring, analysis, and/or visualization purposes. In this context the term “mobile” emphasizes the long range and ambulatory aspects of the device, which provide the patient a wide range (e.g., miles) and independence from in-clinic and at-home stationary antenna systems. This is to be understood as being an improvement on existing technologies, such as Bluetooth-only devices which are limited to a range of approximately 25-30 feet.

MCT devices may sense, save, or send information via Bluetooth and/or upload to webserver. In at least some embodiments, the current disclosure contemplates sensing and/or saving information (for backup), and instantly and automatically uploading to a webserver via LTE/LTE-M1 or WIFI. There is no need for an operator to manage the data transfer eliminating a possible source of delay or error. Instead, expediting the data upload accelerates or reduces the time delay before critical cardiac and other vital sign related events can be diagnosed and action be taken.

As used herein, the term “cardiac” relates to any one or more vital signs that correspond to a function or health of the heart or cardiovascular system. In this context, “telemetry” refers to communication, such as bi-directional (full duplex) data communication both online (live-stream) and offline.

FIGS. 1A-B show a bottom and top view of a mobile cardiac monitoring device 100, which includes a generally cuboid-shaped housing 102 having a top surface 104 disposed away from the patient and a lower surface 106 disposed closer to the patient. Housing 102 may utilize metallic or plastic materials intended for use in rugged, harsh, and demanding environments. In at least some examples, housing 102 will meet IPX7 standards with a goal to function close to IPX8, i.e., attempting to be waterproof for use in and under water, or completely watertight instead of being waterproof or water-resistant. In some examples, small silicone O-rings are disposed around the electrode snap-in contact (receivers) to create a totally water-proof electrical connection from the transmitter to the one-time use skin electrode so divers could be monitored while working under water. Monitoring device 100 may be used in several modes as will be described in greater detail below.

As shown in FIG. 2B, monitoring device 100 may generally include any one or more of a processor 202, a memory 204, a telemetry unit 206, a microphone and/or speaker 208, a GPS sensor 210, an accelerometer 212, a temperature sensor 213, and battery 214. Telemetry unit 206 may be in communication with a remote webserver, mobile, or stationary computing device capable to serve as data collection unit 220.

Monitoring device 100 may include four receivers 110 configured and arranged to allow connection between monitoring device 100 and single-use skin electrode patches. In the example shown, monitoring device 100 is configured to connect or receive four single-use skin electrode patches 150 (FIG. 2A). In this example, the four receivers are disposed in an asymmetric configuration with receivers 110b,110c,110d being disposed generally on three corners of housing 102, and receiver 110a being offset and disposed closer to the medial line of the housing than the other three receivers. In some examples, the asymmetry is used to indicate the transmitter placement on the patient and to ensure proper transmitter placement on the charging station via receivers that snap into plastic holders to ensure mechanical connection to the charging station while the pogo pins are used for USB data up/download and battery charging. Alternatively, the device may be symmetric with the left-side receivers being aligned with the right-side receivers. The number and/or position of receivers may be varied based on the desired usage (e.g., two, three, four, five or more receivers are possible in various configurations).

By way of explanation, all ECGs pick up heart signals through electrodes connected externally to specific locations on the body. The heart signals are generated by the body and have amplitudes of a few millivolts. The placement of the electrodes in specific locations allow the heart's electrical activity to be viewed from different angles, each of which is displayed as a channel on an ECG printout. Each channel represents the differential voltage between two of the electrodes, or the differential voltage between one electrode and the average voltage from several electrodes. The different combinations of electrodes allow more channels to be displayed than there are electrodes. The number of channels define the number of ECG traces that can be shown simultaneously plus showing the other parameters such as the respiratory curve or skin temperature curve or 3-axis body motion activity curve as a graphic. The ECG channels may be commonly referred to as “leads,” so that a 12-lead ECG device has 12 separate channels displayed graphically. The number of channels may vary from 1 to 12 depending on the application. Using a four-receiver configuration such as that shown in FIG. 1A, up to six leads, typically what is called the limb leads (Einthoven & Goldberger) may be monitored and other leads (e.g., chest leads) may be derived through the addition of differently configured chest adapter with more than 4 receiver contacts to measurement or calculation of a 12-lead ECG from the obtained data.

Monitoring device 100 may be used as a diagnostic tool to obtain diagnostic resting ECG with 1, 2, 3, 6, or 12 leads derived and measured. Additionally, monitoring device may be used as a Holter and Event monitor with 24-, 48-, or 96-hours recording time, that sends data either live online or up to 14 days offline while saving data on memory 204 for later download (e.g., via USB). Monitoring device 100 may also be used as an ICU telemetry monitor with 24-, 48-, 96-hours recording time, sending data live online when used inside clinical environments. Note that battery 214 may require more frequent recharging when used solely on WLAN than used solely in Bluetooth or LTE connection mode. Monitoring device 100 may, additionally or instead, be used as (MCT) outpatient monitoring device, sending status reports at predefined intervals via LTE Cat1 module via cell phone network to telemedicine provider or caregiver, and sending alerts immediately when detecting critical cardiac events. Monitoring device 100 may, additionally or instead, be configured to upload complete Holter-like recordings via LTE-M1 network, instantly (live-streaming) in high resolution continuously and at certain intervals, for in-depth analysis and to save time compared to conventional systems that can only upload data after the recording has been finished. Instant upload ensures critical cardiac data can be analyzed with very little latency which reduces the response time when a critical cardiac event is detected.

It least some examples, monitoring device 100 may provide a readout of system health status and may continuously and/or automatically connect via LTE or WLAN. The monitoring device may install new firmware in the background automatically without user input. The monitoring device 100 may also check lead placement and recording quality when a new recording is started, and/or may automatically notify a patient or a caregiver that lead position placement is suboptimal and needs to be improved by relocating the transmitter contact receivers on the patient's body. The device may also restart the recording process after adjustment of lead placement if the quality of the data is deemed to be insufficient, which eliminates the need to inspect low-quality data by the analyst or automated analysis software.

In some examples, the system may include firmware and a programming mode configured to perform at least one of the following steps: (1) default with all channels in recording; (2) selected channels and functions turned off to save battery and reduce data volume; (3) selected radios turned off to save battery or operating cost for user (e.g. LTE roaming costs vs free WLAN, etc.); (4) all or some of the channels turned off (i.e., no sensing functions) and device used as a hub device only to bridge and transfer data received from other sensor devices connected to the transmitter for upload to a webserver or display locally on a BT or WLAN connected device using a viewer/analysis software; (5) enable different product variants at different sales price levels; and/or (6) possibly breakdown system configuration using separate SKU part numbers for identification. In some examples, two fingers on snap-in contacts may be used to turn-on the device 100 from a battery energy saving sleep mode, triggering a mini-ECG signal, which eliminates the necessity for a physical on/off button. The absence of a physical on/off button makes it easier to waterproof the transmitter casing but also eliminates the risk that a patient can accidentally (or not) turn off the transmitter. Conventional other brand transmitters or recorders also use physical buttons to set cardiac event markers (patient is instructed to press a button when feeling unwell which sets a time stamp in the digital recording that the software or analyst can later find easily) are not easy to use when the transmitter is worn underneath a shirt or clothing. Alternatively, the accelerometer may be used to trigger the device on/off by asking patient to move the transmitter (e.g., rotate/roll the device through 360 degrees of rotation) or to set an event marker by tapping once or twice on the recorder). The microphone may also be used to turn the device on/off or set cardiac event marker by saying a predetermined command (e.g., “turn on,” or “stop measuring.”).

In addition to these heart signals, the built-in, highly precise accelerometer 212 may measure the position of the patient in all three-axes relative to the ground and can detect if the patient is standing or laying down. Accelerometer 212 may also detect sudden motion (e.g., falls, jumps, running), and other activities that can be used to identify and eliminate artifacts in the ECG data. In at least some examples, accelerometer 212 may be used to turn on/off the transmitter by measuring motion and sending the device to sleep to save battery when no motion is detected. In at least some examples, accelerometer 212 may be used to assist and/or control proper lead placement by sending ECG and acceleration data to a clinic/caregiver, who in turn can provide the patient with instructions (written or audible) on how to hold or manipulate the chest adapter or how to place the electrode patches (e.g., “turn the adapter upside down,” or “rotate the adapter counterclockwise 45 degrees”). The accelerometer 212 may be activated first and it may initiate an ECG recording when it senses that the patient is at rest to avoid artifacts. The accelerometer 212 may also detect and measure coughing (i.e., it may sense a sudden movement or “violent jerk” that it can correlate to a cough based on amplitude, duration, periodicity and the like or suitable combinations thereof, and identify an individual or condition from the coughing pattern (e.g., for use with Covid monitoring). The accelerometer may also be used in drug rehabilitation to monitor movement or breathing patterns of recovering drug addicts or Covid19 patients that spontaneously stop breathing or begin labored breathing. In some examples, accelerometers 212 is configured to achieve up to 500 Hz or 1000 Hz resolution, which is a much higher resolution compared to conventional motion activity trackers found in Fitbits, iPhones etc.

Embedded GPS sensor 210 may enable precise location of the patient which, when shared with emergency responders, makes for faster arrival of help in emergency situations (e.g., after detecting a critical cardiac signal or other event). The built-in microphone and/or loudspeaker 208 may enable communication with the patient or caregiver to assess a medical situation in emergencies. Monitoring device 100 may automatically detect and absorb AED (external defibrillator) shocks up to 5000V, and/or may include NFC to readout regulatory relevant FCC identification numbers, e.g., GSM serial numbers, Bluetooth numbers, WLAN numbers, product identification number, and firmware revisions.

Monitoring device 100 may perform certain calculations using processor 202. For example, processor 202 may calculate heart rate from ECG data. Processor 202 may also calculate or identify critical cardiac events such as pauses, tachyarrhythmia, bradycardia, extrasystoles, and VPCs based on comparing measured values to predetermined thresholds or models stored in memory 204. Processor 202 may also calculate respiratory values from impedance measurements between the electrode receivers using a software algorithm to amplify the impedance readout to enable short distance between the receivers.

Monitoring device 100 may, additionally or alternatively, sense myoclonic jerks via muscle spasms and changes in electrical conductivity on the skin surface between electrode patches. The monitoring device 100 may also include a skin temperature sensor to measure changes in the body surface temperature relative to room temperature to identify fevers and infections. In some examples, device 100 may also connect or pair via Bluetooth or WLAN with an external room temperature sensor for reference.

Additionally, when connected to an external medical sensor via cable or wirelessly via Bluetooth, device 100 may receive and process additional parameters such as blood oxygen, blood, pressure, and/or blood glucose received from such external sensors continuously and process the received data in combination with the cardiac ECG data retrieved by the transmitter directly from the patient so that they can be send (streamed live) and used for ICU vital sign monitoring.

Using processor 202, monitoring device may calculate RR, ST, QT, and other cardiac relevant data from the ECG signal. Alternatively, some or all of these calculations may be performed with another device after the raw data is communicated via the telemetry unit. Embedded AI functions may be used to process patient data prior to sending the data to the cloud or connected display device. The monitoring device may communicate (i.e., send or receive) data using proprietary software installed on proprietary web servers and/or telemedicine server platforms from clients. Monitoring device 100 may also be used to detect incoming epileptic events via myoclonic jerk detection, detect labored and critical breathing behavior in Covid19 patients and drug addicts and it can serve as a hub device or bridge to transmit data from third party sensors to the cloud, and/or to serve as a hub device to connect to one or more proprietary external sensors via cable or wirelessly via Bluetooth, and receive and process additional cardiac and vital sign parameters such as blood oxygen, blood, pressure, and/or blood glucose continuously.

Any of the measured and/or calculated data may be stored in memory 204 or transmitted to an external device. In at least some examples, telemetry unit 206 allows for sending and/or receiving data from the monitoring device 100. The data may be sent, for example, to one or more mobile devices using iOS, Android, Linux or Windows-based operating systems via proprietary and custom designed mobile applications. Proprietary software, such as CardioExplore™ or VMX™, VM00™, may also be installed on client computers to access this data.

In some embodiments, monitoring device 100 may include a telemetry unit 204 that includes one or more of a built-in NFC, Bluetooth, WLAN-radios, and two LTE cell phone systems, including antennas to exchange data with the outside world. In at least some examples, a WLAN module may include a reduced power consumption mode enabling up to 8 hours or more of WiFi in ICU mode (continuous and instant live streaming of cardiac and vital sign parameters) using very miniaturized batteries with 1200 mAh or more capacity between recharging). The WLAN module may be USB-compatible, and may have purposely slow and reduced to 3-to-4-hour battery recharging time which increases the overall use of the transmitter to expected 5-7 years of operational life. Optionally a fast charger may be used. The telemetry unit may include one or more antennas. In at least some examples, two antennas or modules are disposed next to each other to provide two forms of LTE communication that can be operated in parallel or signal transmission can be switched from one to the other module depending on programming and use conditions. These modules may include a first LTE CAT-MI module with large bandwidth for high-speed data transfer of very large sized, complete ECG data sets (e.g., for use in Holter analysis), and a second LTE CAT-I module for intermittent data burst transmissions only to report system status and alerts such as cardiac alarms) to save battery life. Multiple SIM card variants may be used to maximize versatility in order to function in different parts of the world and territories. For example, a SIM card with voice functionality may be used to enable speech communication (e.g., for calling a physician, hospital or emergency operator). Additionally, an M2M chip variant (e.g., the LTE-MI) with no speech functionality, but high data transmission may be used to send and/or receive the data and provide independence from a specific carrier network. Using this combination, LTE with voice functionality may enable clinic, doctor, and caregiver to establish contact to the patient and assess the situation while retrieving medical data. Meanwhile, data may be transmitted instantly from patient to clinic, where an emergency response may be initiated. Thus, the cardiac condition of the patient may be communicated with no wait time and instant data availability. In such configurations, either one or both LTE antennas may be oriented facing away from the patient (i.e., toward top surface 104) to reduce FCC emissions rate.

In some examples, the same receivers are capable of mechanically and/or electrically coupling to multiple sensing modalities including one or more of single-use skin electrode patches, lead cables, or chest adapter plates (described in greater detail below). Through the use of the accessories being coupled to the receiver, monitoring device 100 may be mechanically and/or electrically coupled to the patient's skin and acquire electrical data that the built-in software algorithms process into ECG and other medically useful data.

Monitoring device 100 may also include one or more pins 115 (e.g., metallic contacts, also called pogo pins) to provide mechanical and/or electrical connection between the monitoring device 100 and a chest adapter, docking and/or charging station. Monitoring device 100 may be grouped as part of a kit or platform that includes monitoring device 100, one or more single position or four-position docking/battery charging stations, a chest-probe adapter to derive resting diagnostic ECG, lead cables to derive an ECG (e.g., 6 or 12-lead ECG), a USB data download cable for single position docking/charger station, a power adapter for four position docking/charger station with cables, firmware to operate the monitoring device 100, and/or a configuration file to control parameter settings in the monitoring device 100.

As will be understood from the preceding description, the mobile cardiac monitoring device includes several optional and/or interchangeable components that allow it to serve different functions. Thus, the device may be deemed an “all-in-one” device as it may be used to combine and/or replace three systems: (1) diagnostic resting ECG, (2) Holter and ICU and MCT monitor with cables and (3) Holter and ICU monitor without cables (e.g., through the use of skin adhesive electrode patches only).

Turning to FIGS. 3-5, one example of an adapter 300 is shown, to be used with monitoring device 100. Specifically, monitoring device 100 was previously described in its long-term Holter and ICU telemetry and MCT configurations where it is coupled, via receivers 110, with four single-use skin electrode patches 150, such as AMBU® BlueSensor skin adhesive electrodes or patches of other commercially available or of proprietary use (See, FIGS. 1A-2). When only short ECG recordings (approximately 10-60 seconds) for diagnostic ECG evaluations are needed, the operator can either attach traditional ECG lead cables or snap the backside electrode receivers from the monitoring device into a chest adapter 300 (FIG. 3A).

Chest-probe adapter 300 may include a substantially planar butterfly-shaped plate 305 having a lower surface 302 and an upper surface 304, the lower surface being positioned closer to patient tissue. In at least some examples, plate 305 may be formed of a printed circuit board. In some examples, plate 305 includes a rigid printed circuit board to hold the connector balls and to embed the printed or etched metal conductor traces that connect the receiver snap-ins on the top side (that go into the transmitter) with the metal balls that go to the patient. Plate 305 may have a curved upper edge 310 and a generally straight lower edge 312 disposed opposite the curved upper edge. Plate 305 may also define a pair of curved side edges 314 opposing one another. The shape and size of the adapter, and specifically plate 305, may allow it to be operated using only one hand. Plate 305 may mate with receivers 110 of monitoring device 100 via connectors 320 that align with the receivers. In at least some examples, monitoring device 100 may be releasably coupleable to plate 305 via clips, press fits, frictional interference or other known methods.

Chest adapter 300 may include four integrated ball-shaped contact electrodes 330 positioned on extreme corners of plate 305 and configured to be pressed against the skin (or fur in case of animals), best shown in FIGS. 4A-C. In some examples, contact electrodes 330 may be made of stainless steel or other suitably conductive materials. Although each contact electrode is shown in the shape of a ball, electrodes may alternatively be hemispherical or dome-shaped, with the curved part of the electrodes being disposed away from the plate and toward the patient. As used herein, the term “ball” will be understood that include geometrically spherical configurations as well as ovoid and other evenly or unevenly rounded bodies. The ball-shaped electrodes may create a compliant contact pattern on the surface of the patient that is well tolerated and pain-free (i.e., the contacts do not have sharp edges that dig into the patient's skin when pressed against it). Additionally, the ball-shaped electrodes may create a large surface area per contact point, which reduces the dependence on excellent skin conductivity and improves the signal capture quality of the adapter. Using adapter 300, the distance between ball-shaped contact electrodes 330 to each other is short compared to traditional ECG lead limb cables, and a smart algorithm may be used to amplify the captured signal without amplifying the background noise. In some examples, plate 305 includes a printed circuit board that is used to hold and connect ball-shaped contact electrodes 330 via screws 331 that are received and mount on the inside of the electrodes 330 (FIG. 3B). In some examples, one or more metallic connectors 316 on a top surface of the adapter may be configured to mate and couple with receivers 110 on the bottom portion of the bottom surface of monitoring device 100 (FIG. 3C). In at least some examples, four protruding connectors 316 are used, and these are configured and arranged to align with the receivers 110, which may be symmetric or asymmetric as previously noted.

In at least some examples, the electrical voltage difference between the four contact electrodes 330 is measured and computed into an ECG signal through monitoring device 100. In this example, the four contact electrodes 330 represent the electrode contacts of the limb leads and derive the 6-leads of an Einthoven/Goldberger ECG. The placement and orientation of chest adapter 300 on the chest or ribcage may not derive a “normalized” ECG. Thus, software on the display may be used to correct for the “misaligned” ECG signal recorded using the chest adapter and after processing display a corrected “normalized” ECG cardiogram. In this context, the accelerometer 212 of monitoring device 100 may be used to ascertain the position of the device and relay that with the data as an indication of the adapter orientation. In use, the operator may hold the chest adapter 300 in their hand and press the four round contact electrodes 330 against the chest or ribcage of the patient (human in FIG. 5A or dog in FIG. 5B) in lieu of traditional lead cables and derive an ECG.

In some embodiments, the built-in LTE and WIFI allows for forgoing the intermediate step of mobile or stationary device data transfer, and the device may send directly information into the cloud. This data may be sent live/instantly with very little latency, and browser-based software may display and automatically analyze the incoming cardiac and other data. User may be able to see the information in a web browser window or receive an SMS message and/or email with alerts and reports.

In some embodiments, the LTE, WIFI and/or Bluetooth radio may be used to receive data from a secondary, external medical sensors (e.g., blood oxygen, pressure, glucose) when in hub mode, and send them combined or individually via LTE or WIFI to the webserver or directly connected computers and mobile devices. This may be possible due to the presence of both LTE and WIFI.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A system comprising:
a monitoring device including a housing having a top surface and a bottom surface, the monitoring device having a processor and a memory and being configured and arranged to measure cardiac data, the monitoring device having a plurality of receivers configured to accept at least one of (i) a plurality of skin electrode patches or (ii) an adapter having integrated contact electrodes.

2. The system of claim 1, wherein the plurality of receivers includes four receivers, the four receivers being configured and arranged to electromechanically removably couple to the plurality of electrode patches, or to four protruding connectors disposed on the adapter and in a configuration that aligns with the four receivers.

3. The system of claim 1, wherein the plurality of receivers is asymmetric.

4. The system of claim 1, wherein the monitoring device further includes a telemetry unit having a first LTE CAT-I module and a second LTE CAT-MI module, a WLAN module and a Bluetooth module disposed on a circuit board.

5. The system of claim 1, wherein the monitoring device further includes one or more pins for coupling to a charging dock.

6. The system of claim 1, wherein the monitoring device further includes an accelerometer, a GPS module and a skin temperature sensor.

7. The system of claim 6, wherein the accelerometer is configured and arranged to conserve battery life when no motion is detected for a predetermined duration of time.

8. The system of claim 6, wherein the accelerometer is configured and arranged to identify the orientation of the adapter, and wherein the accelerometer is configured and arranged to apply a time stamp when manually tapped by the patient.

9. The system of claim 1, wherein the monitoring device further includes a microphone and a speaker.

10. The system of claim 1, wherein the monitoring device further includes at least one antenna that faces the top surface of the monitoring device.

11. The system of claim 1, wherein the adapter includes a substantially planar plate comprising a printed circuit board and the integrated contact electrodes are rounded.

12. The system of claim 11, wherein the integrated contact electrodes comprise a metal.

13. The system of claim 11, wherein the integrated contact electrodes are disposed at corners of the substantially planar plate.

14. The system of claim 1, wherein the integrated contact electrodes are spherical.

15. The system of claim 1, wherein the monitoring device is releasably coupleable to the adapter via an electromechanical interference fit.

16. A method of treating a patient comprising:
providing a monitoring device including a housing having a top surface and a bottom surface, the monitoring device having a processor and a memory and being configured and arranged to measure cardiac data, the monitoring device having a plurality of receivers on the bottom surface;
selecting between a plurality of single-use skin adhesive electrode patches or an adapter having integrated contact electrodes based on the intended use; and
releasably coupling one of the plurality of single-use skin electrode patches or the adapter to the plurality of receivers of the monitoring device.

17. The method of claim 16, wherein the adapter includes a substantially planar plate and the integrated contact electrodes are rounded, and further comprising the step of holding the adapter and pushing the integrated contact electrodes against a patient body.

18. The method of claim 16, further comprising providing an accelerometer and a GPS module within the monitoring device.

19. The method of claim 18, further comprising conserving battery life when no motion is detected by the accelerometer for a predetermined duration of time.

20. The method of claim 18, further comprising identifying an orientation of the adapter via the accelerometer.
